(19) United States
(12) Patent Application Publication
(10) Pub. No.: US 2007/0202134 A1
(43) Pub. Date: Aug. 30, 2007

(54) Muc1 Antagonist Enhancement of Death Receptor Ligand-Induced Apoptosis

(76) Inventors: Donald W. Kufe, Wellesley, MA (US); Surender Kharbanda, Natick, MA (US)

Correspondence Address: FULBRIGHT & JAWORSKI L.L.P., 600 CONGRESS AVE., SUITE 2400, AUSTIN, TX, 78701, US

(21) Appl. No.: 10/598,295

(22) PCT Filed: Feb. 22, 2005
(86) PCT No.: PCT/US05/05508

§ 371 Date: Apr. 5, 2007

Related U.S. Application Data
(60) Provisional application No. 60/547,010, filed on Feb. 23, 2004.

(51) Int. Cl. A61K 39/00; A61P 43/00; C12N 5/06; C12N 5/08

(52) U.S. Cl. 424/277.1; 435/375

(57) ABSTRACT

The present invention relates to methods of enhancing death receptor-induced apoptosis in MUC1 expressing cells comprising contacting the MUC1 expressing cells with an effective amount of a MUC1 antagonist.

The present invention claims benefit of priority to U.S. Provisional Ser. No. 60/547,010 filed Feb. 23, 2004, the entire content of which is hereby incorporated by reference.

### BACKGROUND OF THE INVENTION

1. Field of the Invention

The present invention relates generally to the field of cancer and other therapeutic therapies wherein benefit is derived from cell death ligand-induced apoptosis. More specifically, the present invention relates to use of MUC1 antagonists to enhance death receptor ligand induced apoptosis.

2. Background

The apoptotic response of cells is induced by extrinsic and intrinsic pathways that activate the caspase family of cysteine proteases. The extrinsic apoptotic pathway is activated by ligand stimulation of the tumor necrosis factor The apoptotic response of cells is induced by extrinsic and intrinsic pathways that activate the caspase family of cysteine proteases. The extrinsic apoptotic pathway is activated by ligand stimulation of the tumor necrosis factor (TNF) family of death receptors. Activation of caspase-8 by death receptor signaling results in cleavage of procaspase-3 (Boldin et al., 1996; Muzio et al., 1996; Stennicke et al., 1998). Caspase-8 also cleaves Bid, a proapoptotic member of the Bcl-2 family, and thereby stimulates release of mitochondrial cytochrome c to the cytosol (Li et al., 1998; Luo et al., 1998). Activation of the intrinsic pathway by diverse Bid-independent stress signals is also associated with the release of mitochondrial cytochrome c (Kluck et al., 1997; Liu et al., 1996; Yang et al., 1997). In the cytosol, cytochrome c forms a complex with Apaf-1 and activates caspase-9 (Li et al., 1997; Srinivasula et al., 1998). Like caspase-8, caspase-9 can directly activate caspase-3 (Li et al., 1997). In turn, caspase-3 cleaves multiple proteins, which when inactivated or activated by cleavage contribute to the induction of apoptosis. Protein kinase Cd (PKCd) is one such caspase-3 substrate that is cleaved to a catalytically active fragment, the expression of which is sufficient to induce apoptosis (Emoto et al., 1995). Many genotoxic anti-cancer drugs induce apoptosis by activation of the intrinsic pathway (Herr and Debatin, 2001; Kroemer and Reed, 2000). Moreover, resistance to cytotoxic anti-cancer agents is often associated with defects in the intrinsic pathway (Bunz, 2001; Datta et al., 1995).

The human DF3/MUC1 transmembrane glycoprotein is expressed on the apical borders of normal secretory epithelial cells (Kufe et al., 1984). By contrast, transformation of epithelia to carcinomas is associated with marked overexpression of MUC1 throughout the entire cell membrane (Kufe et al., 1984). MUC1 is expressed as a cell surface heterodimer that consists of N-terminal (N-ter) and C-terminal (C-ter) subunits which form a stable complex following cleavage of a single MUC1 polypeptide (Ligtenberg et al., 1992). The >250 kDa N-ter ectodomain contains variable numbers of 20 amino-acid tandem repeats that are extensively modified by O-linked glycans (Gendler et al., 1988; Siddiqui et al., 1988). The ˜20-25 kDa C-ter, which anchors the N-ter to the cell surface, consists of a 58 amino-acid extracellular region, a 28 amino-acid transmembrane domain and a 72 amino-acid cytoplasmic domain (CD). The MUC1-CD is phosphorylated on Y-46 by the epidermal growth factor receptor (EGFR), c-Src (Li et al., 2001; Li et al., 2001a) and Lyn (Li et al., 2003). Other studies have shown that MUC1-CD is phosphorylated on S-44 by glycogen synthase kinase 3b (GSK3b) (Li et al., 1998b) and on T-41 by PKCd (Ren et al., 2002). Phosphorylation on Y-46 and T-41 induces binding of MUC1-CD with the Wnt effector, b-catenin (Li et al., 2001; Li et al., 2001a; Ren et al., 2002). Conversely, GSK3b-mediated phosphorylation of S-44 decreases the interaction of MUC1-CD and b-catenin (Li et al., 1998b). These findings have indicated that MUC1-CD functions in integrating signals from the EGFR and Wnt pathways.

Overexpression of MUC1 confers anchorage-independent growth and tumorigenicity of rodent fibroblasts and human epithelial cells (Li et al., 2003c; Ren et al., 2002). Other work has shown that, in addition to localization at the cell membrane, the MUC1 C-ter is expressed in nuclear complexes with b-catenin (Li et al., 2003a; Li et al., 2003b; Li et al., 2003c). Moreover, treatment of cells with heregulin (HRG), which activates ErbB2-4, is associated with targeting of MUC1 C-ter to the nucleolus in complex with g-catenin (Li et al., 2003a). These observations have indicated that the function of MUC1 as a transforming protein may be mediated by regulating gene expression.

### SUMMARY OF THE INVENTION

The present invention relates to methods of enhancing death receptor-induced apoptosis in MUC1 expressing cells comprising contacting the MUC1 expressing cells subject to death-receptor-induced apoptosis with an effective amount of a MUC1 antagonist. In some embodiments, the MUC1 expressing cells are MUC1 expressing cancer cells. In some embodiments the death receptor-induced apoptosis is Fas-induced apoptosis or is a TRAIL receptor-induced apoptosis.

In some embodiments, the MUC1 antagonist is an antisense polynucleotide or a siRNA polynucleotide or a MUC1 ligand trap molecule, or an inhibitor of the binding of MUC1 to a PDZ domain.

In one aspect of the present invention, the MUC1 expressing cells subject to the method of the invention are within a patient wherein the patient is in need of treatment comprising induction of death receptor-induced apoptosis cell death of the MUC1 expressing cells.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Depiction of SDS-PAGE and immunoblotting with indicated antibodies of mitochondrial fractions from HCT116/vector-A, HCT116/MUC1-A and HCT116/MUC1(Y46F)-A cells.

FIG. 2: Depiction of SDS-PAGE and immunoblotting with indicated antibodies of mitochondrial fractions from HCT116/MUC1-A and HCT116/MUC1(Y46F)-A cells that had been treated with heregulin (HRG) for the indicated times.

FIG. 3: Summary of ciplatin (CDDP) induced apoptosis in HCT116/vector-A, HCT116/MUC1-A and HCT116/MUC1(Y46F)-A cells wherein cells were incubated with 100 μM CDDP for 24 hr then analyzed for sub-G1 DNA.

FIG. 4: Summary of apoptosis induced in both A and B clones of HCT116/vector, HCT116/MUC1 and HCT116/MUC1(Y46F) cells when left untreated (open bars) or treated with 100 μM CDDP for 24 hr (solid bars). The results are presented as percentage apoptosis (mean±SD of three independent experiments) as determined by analysis of sub-G1 DNA.

FIG. 5: Summary of apoptosis induced in both A and B clones of HCT116/vector, HCT116/MUC1 and HCT116/MUC1(Y46F) cells when left untreated (open bars) or treated with 70 μM etoposide for 48 hr (solid bars). The results are presented as percentage apoptosis (mean±SD of three independent experiments) as determined by analysis of sub-G1 DNA.

FIG. 6: Summary of apoptosis induced in both A and B clones of HCT116/vector, HCT116/MUC1 and HCT116/MUC1(Y46F) cells when left untreated (open bars) or treated with 20 ng/ml TNF-α and 10 ng/ml cyclohexamide (CHX) for 12 hr (solid bars). The results are presented as percentage apoptosis (mean±SD of three independent experiments) as determined by analysis of sub-G1 DNA.

FIG. 7: Summary in left panel of apoptosis induced in HCT116/vector-A, HCT116/MUC1-A and HCT116/MUC1(Y46F)-A cells when left untreated (open bars) or treated with 100 ng/ml TRAIL for 14 hr (closed bars). Summary in right panel of apoptosis induced in HCT116/MUC1 (Y46F)-A cells when treated with 100 ng/ml TRAIL and/or 10 μM CHX as indicated for 14 hr. The results are presented as percentage apoptosis (mean±SD of three independent experiments) as determined by analysis of sub-G1 DNA.

### DETAILED DESCRIPTION

I. MUC1 Downregulation of Death Receptor-Ligand Induced Apoptosis

MUC1 is an oncoprotein that attenuates the apoptotic response to DNA damaging agents and confers resistance to genotoxic anticancer agents (US patent application, Kufe and Ohno, “MUC1 Extracellular Domain and Cancer Treatment Compositions and Methods Derived Therefrom,” filed Feb. 13, 2004, herein incorporated by reference). In addition to blocking activation of the intrinsic apoptotic pathway, expression of MUC1 attenuates TRAIL induced apoptosis. Thus, MUC1 expression also downregulates death receptor ligand-induced apoptosis. Treatment of MUC1 expressing cells with an effective amount of a MUC1 antagonist provides a mechanism to relieve the downregulation of death receptor ligand-induced apoptosis. This is beneficial in the treatment of MUC1-expressing cells wherein it is desirable to stimulate apoptosis associated with the death receptor pathway.

II. Intrinsic and Extrinsic Apoptotic Mechanisms

Two main signaling pathways initiate the apoptotic program in mammalian cells. The cell-extrinsic pathway triggers apoptosis in response to engagement of death receptors by their ligands. Ligand-induced activation of cell-surface death receptors leads to rapid assembly of a death-inducing signaling complex (DISC) and activation of the apoptosis-initiating proteases caspase-8 and caspase-10. These caspases activate caspase-9 that in turn activates caspase-3, -6, and -7. The extrinsic-cell pathway is a mechanism used by NK and cytotoxic T lymphocytes to trigger apoptosis in tumor cells and virus infected cells.

The cell intrinsic pathway triggers apoptosis in response to DNA damage, defective cell cycle, hypoxia, loss of survival factors and other types of cell stress. This pathway involves activation of the pro-apoptotic arm of the BCL2 gene family that engage the mitochondria to cause the release of apoptogenic factors such as cytochrome c and SMAC/DIABLO into the cytosol (Adams et al., 1998; Hunt & Evans, 2001). In the cytosol, cytochrome c binds to adaptor APAF1, forming an apoptosome that activates caspase-9 that in turn, as in the extrinsic pathway, activates caspase-3, -6, and -7. SMAC/DIABLO promotes apoptosis by binding to inhibitor of apoptosis proteins and preventing these factors from attenuating caspase activation (Du et al., 2000; Verhagen et al., 2000). Most chemotherapy agents and irradiation trigger tumor-cell apoptosis through the cell-intrinsic pathway, as an indirect consequence of causing cellular damage.

The two apoptotic pathways are interconnected. Death receptors can activate the intrinsic pathway by caspase-8-mediated cleavage of the apical pro-apoptotic BCL2 family member BID (Li et al., 1998; Luo et al., 1998; Gross et al., 1999). BID interacts with the pro-apoptotic BCL2 relatives BAX and BAK, which cause release of mitochondrial cytochrome c and SMAC/DIABLO, activating caspase-9 and -3. This amplifies apoptosis induction through the intrinsic pathway. In some cell types, commitment to apoptosis requires amplification of the death-receptor signal by the intrinsic pathway (Scaffidi et al., 1999).

III. Death Ligands and Receptors

A subset of the tumor necrosis factor (TNF) family are involved in initiating a cell death signaling cascade upon binding to the appropriate member of the TNF receptor (TNFR) family, the latter being referred to as the “death receptor family.” Death receptor ligands includes FasL (APO1L or CD95L) and TNF-related apoptosis-inducing ligand (TRAIL or APO2L).

TRAIL selectively induces apoptosis of a variety of tumor cells and transformed cells, but not most normal cells, and therefore has garnered intense interest as a promising agent for cancer therapy (Wang & El-Deiry, 2003). TRAIL is expressed on different cells of the immune system and plays a role in both T-cell- and natural killer cell-mediated tumor surveillance and suppression of suppressing tumor metastasis. Four TRAIL receptors have been identified, two death receptors, DR4 (TRAIL-R1) and DR5 (TRAIL-R2) and two decoy receptors DcR1 (TRAIL-R3) and DcR2 (TRAIL-R4) (Pan et al., 1997; Pan et al., 1997a; Walczak et al., 1997; Marsters et al., 1997). Like most other TNF family members, TRAIL form homotrimers that bind three receptor molecules, each at the interface between two of the subunits (Hymowitz et al., 1999). A zinc atom bound by cystines in the trimeric ligand is essential for trimer stability and optimal biological activity (Bodmer et al., 2000). Administration of TRAIL in in vivo murine and primate models, induces tumor regression without systemic toxicity (Ashkenazi et al., 1999; Walczak et al., 1999). TRAIL also induces apoptosis in a variety of cancer cell lines regardless of p53 status. Some mismatch-repair-deficient tumors evade TRAIL-induced apoptosis and acquire TRAIL resistance through different mechanisms. It has been found that Bax is required for TRAIL induced apoptosis of certain cancer cell lines, possibly by allowing release of SMAC/DIABLO (Deng et al., 2002), and Bax inactivation in MMR-deficient tumors can cause resistance to TRAIL (Burns & El Deiry, 2001; LeBlanc et al., 2002). TRAIL treatment in combination with chemo- or radiotherapy enhances TRAIL sensitivity or reverses TRAIL resistance by regulating the downstream effectors (Wang & El-Deiry, 2003). Enhancement of the mitochondrial apoptotic pathway provides a way of increasing sensitivity to TRAIL.

Various recombinant versions of human TRAIL have been generated. One version contains amino acid residues 114-281 of TRAIL fused to an amino-terminal polyhistidine tag (Pitti et al., 1996). A second variant contains amino acids 95-281 fused via the amino terminus to a modified yeast Gal4 leucine zipper which promotes trimerization of the ligand (Walczak et al., 1999). A third version contains residues 95-281 fused to an amino-terminal “Flag” tag. Crosslinking of this tagged protein with anti-flag antibodies enhances its activity against certain cell lines such as Jurkat T leukemia (Bodmer et al., 2000). A fourth recombinant version of residues 114-261 of human TRAIL without any added exogenous sequences may be the current most preferred form for clinical applications (Ashkenazi & Dixit, 1999). This version is the least likely to be immunogenic in human patients. Such soluble recombinant TRAIL proteins are of interest for cancer therapy because they constitute one of the few examples of molecules that kill many transformed cells but not most normal cells (Ashkenazi & Dixit, 1998).

Fas-mediated apoptosis is triggered by FasL, a type II membrane protein that can be proteolytically cleaved to from a bioactive trimer (Kayagaki et al., 1995; Mariani et al., 1995). After FasL has been bound, Fas associates with two specific proteins, Fas-associated death domain (FADD) and caspase-8 to from a death-inducing signal complex (DISC) (Kischkel, 1995). FasL seems to be important for immune surveillance against tumors and NK cells and cytotoxic T cells can use Fas to induce Fas-expressing tumor-cell targets (Nagata, 1997; French & Tschopp, 1999). However, loss of Fas function occurs frequently during human tumor progression, and may reflect transcriptional downregulation of the Fas gene, selective production of alternatively spliced soluble Fas forms, or loss of Fas signaling as a consequence of BCL2, BCL-xL, FAP-1 or FLIP (Jattela et al., 1995; Srinivasan et al., 1998a; Sato et al., 1995; Irmler et al., 1997; Kataoka et al., 1998). Many such tumors also appear to demonstrate constitutive FasL expression that may mediate immune privilege and induce peripheral tolerance through apoptosis of Fas-positive effector T lymphocytes (Griffith et al., 1996; Bellgrau et al., 1995; Milik et al., 1997).

In vivo experiments in murine models have shown that anti-Fas antibodies, FasL-expressing cells and recombinant Fas reduce the growth of transplanted solid tumors. Unfortunately, these agents also cause severe damage to the mouse liver (Timmer et al., 2002). However, enhancement of endogenous Fas-induced apoptosis may be useful as an adjunct therapy with anti-tumor vaccines and also with use of conventional chemotherapeutic agents. FasL may function as an autocrine/paracrine mediator of apoptosis induced by DNA-damaging chemotherapeutic agents (Poulaki et al., 2001).

IV. MUC1 Antagonists

MUC1 antagonists are agents or compounds that decrease the expression of MUC1 or inhibit the transmembrane and/or intracellular signaling of MUC1. MUC1 antagonists include, but are not limited to, the following agents or compounds:

1. Small Molecules

Small molecules that downregulate the expression of MUC1 include the isocoumarin NM-3 (2-(8-hydroxy-6-methoxy-1-oxo-1H-2-benzopyran-3-yl)propionic acid). NM-3 and other 3-yl-isocoumarins suitable to downregulate the expression of MUC1/ECD are disclosed in U.S. Pat. No. 6,020,363, herein incorporated by reference. Other suitable compounds include 2-substituted estradiol compounds such as 2-methoxyestradiol and 2-hydroxyestradiol. These and other suitable estradiol derivatives are disclosed in U.S. Pat. No. 6,239,123, herein incorporated by reference. Other compounds suitable for downregulating MUC1/ECD expression include the oelanae triterpenoids 2-cyano-3,12-dioxoolean-1,9-diene-28-oic (CDDO), CDDO methyl ester (CDDO-Me), imadzole CDDO (CDDO-Im) and the 2-propyl-imidazole CDDO (CDDO-Pr-Im). Methods relating to measuring down regulation of MUC1 by small molecules are provided in U.S. patent application Ser. No. 10/447,839, by Kufe et al, filed May 29, 2003, herein incorporated by reference.

2. Antisense and siRNA

The expression of MUC1 can be downregulated by antisense or by use of siRNA. Suitable compositions and methods are disclosed in U.S. patent application Ser. No. 10/447,839, by Kufe et al, filed May 29, 2003, herein incorporated by reference.

3. Antibodies

MUC1 transmembrane signaling can be inhibited by use of antibodies against the MUC1/ECD. Details of suitable antibodies are provided by U.S. patent application Ser. No. 10/447,839, by Kufe et al, filed May 29, 2003, herein incorporated by reference.

4. Ligand Traps

Wild type MUC1 ligands include dermcidin. Methods and compositions relating to wild type MUC1 ligand traps, such as dermcidin traps, and other modalities of inhibiting the wild type MUC1 ligand-MUC1 interaction are provided in U.S. Provisional Patent Application Ser. No. 60/519,822, Kharbanda et al., filed Nov. 12, 2003, herein incorporated by reference.

5. PDZ Ligand Binding Inhibitors

The MUC1/CD contains a PDZ binding motif and acts as a PDZ ligand, and such interactions facilitate the intracellular signaling by the MUC1/CD. Compositions and methods relating to MUC1-PDZ binding inhibitors are provided by U.S. Provisional Patent Application Ser. No. 60/502,111, Jecminek et al., filed Sep. 11, 2003, herein incorporated by reference.

### EXAMPLES

### Example 1

### MUC1 C-ter Localizes to Mitochondria

Cell culture. Human HCT116 colon carcinoma cells (ATCC, Manassas, Va.) were cultured in Dulbecco's modified Eagle's medium/F12 with 10% heat-inactivated fetal bovine serum, 100 units/ml penicillin, 100 mg/ml streptomycin and 2 mM L-glutamine. Cells were treated with EGF (10 ng/ml; Calbiochem-Novabiochem, San Diego, Calif.), HRG (20 ng/ml; Calbiochem-Novabiochem), cisplatin (CDDP; Sigma), etoposide (Sigma), rhTNF-a (Promega, Madison, Wis.), CHX (Sigma) or rhTRAIL (100 ng/ml; Calbiochem-Novabiochem).

Cell transfections. HCT116 cells were transfected with pIRES-puro2, pIRESpuro2-MUC1 or pIRES-puro2-MUC1(Y46F) as described (Li et al., 2001a). SW480 cells were transfected with pIRES-puro2 or pIRES-puro2-MUC1. Stable transfectants were selected in the presence of 0.4 mg/ml puromycin (Calbiochem-Novabiochem, San Diego, Calif.). Two independent transfections were performed for each vector. Single cell clones were isolated by limiting dilution and expanded for analysis. In other studies, HCT116 cells were transiently transfected with the pEGFP-C1 vector (Clontech) in which MUC1 C-ter was cloned downstream to sequences encoding the green fluorescence protein (GFP).

Immunoblot analysis. Lysates were prepared from subconfluent cells as described (Li et al., 2001a). Equal amounts of protein were separated by SDS-PAGE and transferred to nitrocellulose membranes. The immunoblots were probed with anti-MUC1 N-ter (DF3) (Kufe et al., 1984), anti-MUC1 C-ter (Ab5; Neomarkers, Fremont, Calif.), anti-MUC1 C-ter (rabbit polyclonal DF3E) (Li et al., 2001), anti-MUC1 C-ter (human monoclonal ECD1), anti-b-actin (Sigma), anti-HSP60 (Stressgen Biotechnologies, Victoria, BC, Canada), anti-PCNA (Calbiochem-Novabiochem, San Diego, Calif.), anti-IkBa (Santa Cruz Biotechnology, Santa Cruz, Calif.), anti-calreticulin (Stressgen Biotechnologies; Victoria, BC, Canada), anti-PDGFR (Santa Cruz Biotechnology), anti-cytochrome c (BD PharMingen, San Diego, Calif.), anti-caspase-3 (BD PharMingen), anti-PKCd (Santa Cruz Biotechnology) anti-Smac/DIABLO (Medical & Biological Laboratories, Ltd., Japan) or anti-AIP (Santa Cruz Biotechnology). The immunocomplexes were detected with horseradish peroxidase-conjugated secondary antibodies and enhanced chemiluminescence (ECL, Amersham Biosciences, Piscataway, N.J.). Intensity of the signals was determined by densitometric scanning.

Flow cytometry. Cells were incubated with anti-MUC1 N-ter or control mouse IgG for 1 h at 4° C., washed, incubated with goat anti-mouse Ig fluorescein-conjugated antibody (Jackson Immunoresearch laboratories, West Grove, Pa.) and then fixed in 2% formaldehyde/PBS. Reactivity was detected by FACScan.

Confocal microscopy. Cells cultured on coverslips were incubated in Dulbecco's modified Eagle's/F12 medium containing 100 nM MitoTracker Red CMXRos (Molecular Probes, Eugene, Oreg.) for 20 min at 37° C. After staining, the cells were washed with fresh growth medium, pre-fixed in 3.7% formaldehyde/growth medium for 15 min at 37° C., washed with PBS, permeabilized in PBS containing 0.2% Triton X-100 for 5 min at 25° C., washed with PBS, then post-fixed in 3.7% formaldehyde/PBS for 5 min at 25° C. After several washes with PBS, the cells were blocked with 10% goat serum for 1 h at 25° C., stained with anti-MUC1 C-ter antibody for 1.5 h at 25° C., washed with PBS, incubated with FITC-conjugated secondary antibody (Jackson ImmunoResearch Laboratories, West Grove, Pa.) for 40 min at 25° C., washed with PBS and incubated with 2 mM TO-PRO3 (Molecular Probes) for 10 min at 25° C. After mounting the coverslips, images were captured with a LSM510 confocal microscope (ZEISS) at 1024×1024 pixel resolution. The excitation wavelength for FITC, MitoTracker Red and TO-PRO3 were 488 nm, 543 nm and 633 nm, respectively. Fluorescein fluorescence was captured through a 505- to 530-nm band-pass filter. MitoTracker Red CMXRos fluorescence was collected through a 560- to 615-nm band-pass filter. TO-PRO3 staining was visualized through a 650-nm long-pass filter.

Subcellular fractionation. Purified mitochondria and cytoplasmic lysates were prepared as described (Kumar et al., 2003). Cell membranes were purified from supernatants after sedimentation of nuclei and mitochondria as described (Kharbanda et al., 1996).

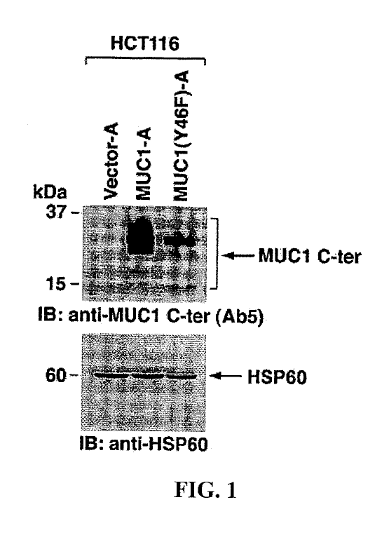

Results. MUC1-negative HCT116 cells were transfected to stably express the empty vector, MUC1 or MUC1(Y46F) mutant. Two clones (A and B) of each were selected from independent transfections. Immunoblot analysis with anti-MUC1 demonstrated no detectable expression of the MUC1-N-ter or C-ter subunits in the vector transfectants. By contrast, MUC1 N-ter expression was similar in cells transfected with MUC1 or MUC1(Y46F). Similar levels of MUC1 C-ter were also found in the MUC1 and MUC1(Y46F) transfectants. To assess whether MUC1 is expressed at the cell membrane, the transfectants were analyzed by flow cytometry with the anti-MUC1 N-ter antibody. In contrast to HCT116/vector cells, MUC1 was detectable on the surface of HCT116 cells expressing MUC1 or MUC1(Y46F). To further define the distribution of MUC1, confocal microscopy was performed with antibodies against the MUC1 N-ter and C-ter. Both subunits were detectable at the cell membrane of the MUC1 transfectants. Unexpectedly, however, MUC1 C-ter, and not N-ter, was also expressed in a pattern that suggested mitochondrial localization. Indeed, colocalization of the MUC C-ter and MitoTracker supported targeting of MUC1 C-ter to mitochondria. By contrast, there was substantially less mitchondrial localization of the MUC1(Y46F) C-ter. Higher magnification and focusing of images within a single HCT116/MUC1 cell showed clear localization of MUC1 C-ter at the cell membrane and with Mitotracker throughout the mitochondrial network. Notably, detection of MUC1 C-ter at the cell membrane is not evident when focusing the confocal microscope of the mitochondria. To confirm these findings, mitochondrial lysates from the transfectants were subjected to immunoblot analyses with anti-MUC1 C-ter. The results demonstrate that the C-ter is detectable in the mitochondrial fraction from HCT116/MUC1, but not from HCT116/vector cells (FIG. 1). Moreover, in concert with the confocal data, mitochondrial localization in the MUC1(Y46F) C-ter was considerably less than that found for the MUC1 C-ter (FIG. 1). Equal loading of mitochondrial lysates were confirmed by immunoblotting for the mitochondrial HSP60 protein. The absence of the N-ter indicated that the mitochondrial fraction was not contaminated with cell membranes. Immunoblot analyses of the mitochondrial lystates with antibodies against the cytosolic IκBα, nuclear PCNA and endoplasmic reticulum-associated calreticulin proteins further indicated that the mitochondria are not significantly contaminated with these subcellular fractions.

To compare MUC-1 C-ter expression at the cell membrane with that in mitochondria, lysates from these fractions were subjected to immunoblot analysis with antibodies directed against the extracellular domain (ECD) and cytoplasmic domain (CD). The results obtained with Ab5 antibody which reacts with the C-terminal 17 amino acids of MUC1 CD, demonstrated similar patterns for MUC1 C-ter expressed at the cell membrane and in mitochondria. Reactivity with Ab5 was observed predominantly at 20-25 kDa. Reactive bands were also observed at approximately 17 and 15 kDa. Immunoblotting with DF3E antibody, which was generated against the VETQFNYKTEAAS motif as described in U.S. patent application Ser. No. 10/447,839, by Kufe et al, filed May 29, 2003, herein incorporated by reference, demonstrated activity with lysates from both the cell membrane and mitochondria. Notably, reactivity of the DF3E antibody with only the 20-25 kDa MUC1 C-ter and the 17 kDa fragments indicated that the 15 kDa fragment, as detected with Ab5, does not contain the DF3E epitope. Another anti-MUC1 ECD antibody, designated ECD1, reacted predominantly with the 20-25 kDa MUC1 C-ter in both the cell membrane and mitochondria. These results suggest that the 17 kDa and 15 kDa fragments represent cleavage within the ECD. As controls, MUC1 N-ter expression was detectable only in the cell membrane fraction and HSP60 expression was restricted to the mitochondrial fraction. Moreover, there was no detectable contamination of the mitochondrial fraction with IkBa, PCNA or calreticulin.

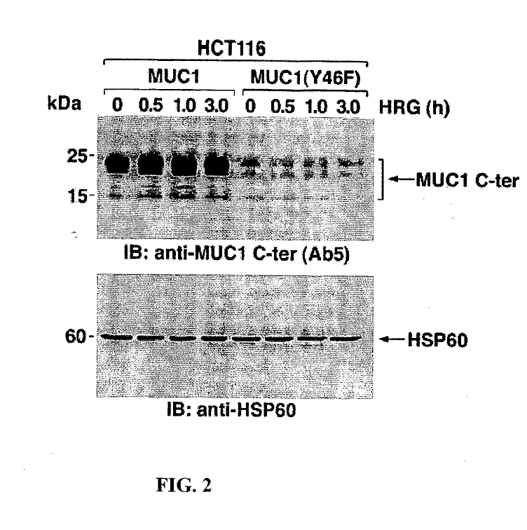

To extend these findings, MUC1 C-ter was expressed with a GFP tag at the N-terminus and assessed mitochondrial localization. Immunoblot analysis of mitochondrial lysates with anti-GFP and anti-MUC1 C-ter confirmed mitochondrial targeting of the GFP-tagged MUC1 C-ter fusion protein. As controls, expression of the platelet-derived growth factor receptor (PDGFR) and HSP60 was restricted to the cell membrane and mitochondrial fractions, respectively. The results of confocal studies also demonstrate colocalization of GFP-MUC1 C-ter with MitoTracker. The transfection efficiency of HCT116 cells is ˜25% under these experimental conditions (Ren et al., 2002). As a control, the prominent pattern of mitochondrial localization was not apparent when expressing GFP alone. These findings collectively demonstrate that MUC1 C-ter localizes to mitochondria. MUC1 C-ter is targeted to the nucleus with β-catenin in cells stimulated with EGF (Li et al., 2001a; Li et al., 2003a). Stimulation of HCT116/MUC1 or HCT116/MUC1(Y46F) cells with EGF, however, had little effect on mitochondrial targeting of MUC1 C-ter. In contrast to EGF, HRG activates ErbB2 in the response of epithelial cells to stress (Vermeer et al., 2003) and targets MUC1 C-ter to the nucleolus (Li et al., 2003). Significantly, HRG treatment for 0.5 h was associated with a 2.3-fold increase in localization of MUC1 C-ter to mitochondria and this response persisted through 3 h (FIG. 2). Moreover, HRG had little effect on mitochondrial localization of MUC1(Y46F) C-ter (FIG. 2). Similar results were obtained in 3 separate experiments. In addition, there was no detectable β-catenin or γ-catenin in the mitochondrial fractions from control or HRG-stimulated cells. These findings indicate that targeting of MUC1 to mitochondria is regulated, at least in part, by HRG-induced signaling and that the Y46F mutation attenuates this response.

### Example 2

### MUC1 Attenuates Cytochrome C Release and Caspase-3 Activation

Methods. Experimental procedures and methods were as described in Example 1.

Results. Treatment of cells with DNA-damaging agents is associated with release of mitochondrial cytochrome c and activation of the intrinsic apoptotic pathway. To determine if mitochondrial localization of the MUC1 C-ter affects cytochrome c release, the HCT116 transfectants were treated with cisplatin (CDDP). Treatment of HCT116/vector cells with CDDP was associated with increased levels of cytosolic cytochrome c. Notably, expression of MUC1 significantly attenuated the release of cytochrome c. By contrast, expression of MUC1(Y46F) was ineffective in blocking CDDP-induced cytochrome c release. Similar results were obtained in the other separately isolated B clones. Release of cytochrome c in the response to genotoxic stress is associated with activation of caspase-3 and cleavage of PKCδ (Emoto et al., 1995). To assess the effects of MUC1 on caspase-3 activation, CDDP-treated cells were analyzed for cleavage of pro-caspase-3. The results demonstrate that, compared to HCT116/vector cells, MUC1 attenuates CDDP-induced activation of caspase-3. Cleavage of pro-caspase-3 in CDDP-treated HCT116/MUC1(Y46F) cells was similar to that in HCT116/vector cells. In concert with these results, caspase-3-mediated cleavage of PKCδ was attenuated in CDDP-treated HCT116/MUC1, as compared to HCT116/vector and HCT116/MUC1(Y46F), cells. Smac/DIABLO is a mitochondrial protein that induces caspase-dependent cell death by interacting with inhibitor of apoptosis proteins (IAPs) and blocking their caspase inhibitory activity (Du et al., 2000; Verhagen et al., 2000). To determine if MUC1 attenuates release of Smac/DIABLO, HCT116/vector, HCT116/MUC1 and HCT116/MUC(Y46F) cells were treated with CDDP for 24, 48 and 72 h, and cytosolic lysates were subjected to immunoblot analysis. The results demonstrate that, like cytochrome c, release of Smac/DIABLO is attenuated in HCT116/MUC1, as compared to HCT116/vector and HCT116/MUC1(Y46F) cells. In addition, MUC1 attenuated release of the mitochondrial caspase-independent death effector, apoptosis-inducing factor (AIF) (Susin et al., 1999), as compared to that in cells expressing the vector or MUC1(Y46F). CDDP treatment of HCT116/vector and HCT116/MUC1(Y46F) cells for 72 h was associated with >90% cell death and decreases in the β-actin signals used as a control for loading. By contrast, treatment of HCT116/MUC1 cells with CDDP for 72 h was associated with cessation of cell growth and <30% cell death. These findings indicate that mitochondrial localization of MUC1 C-ter attenuates DNA damage-induced activation of the intrinsic apoptotic pathway.

### Example 3

### MUC1 Blocks DNA Damage- and TRAIL-Induced Apoptosis

Methods. Apoptotic cells were quantified by analysis of sub-G1 DNA and TUNEL staining. To assess sub-G1 DNA content, cells were harvested, washed with PBS, fixed with 80% ethanol, and incubated in PBS containing 20 ng/ml RNase (Roche) for 60 min at 37° C. Cells were then stained with 40 mg/ml propidium iodide (Sigma) for 30 min at room temperature in the dark. DNA content was analyzed by flow cytometry (EPICS XL-MCL, Coulter Corp.). Apoptotic cells with DNA fragmentation were detected by staining with the In Situ cell death detection kit (TUNEL; Roche Applied Science) and visualized by confocal microscopy (ZEISS LSM510). After staining, cells were analyzed by flow cytometry. Other experimental procedures and methods were as described in Example 1.

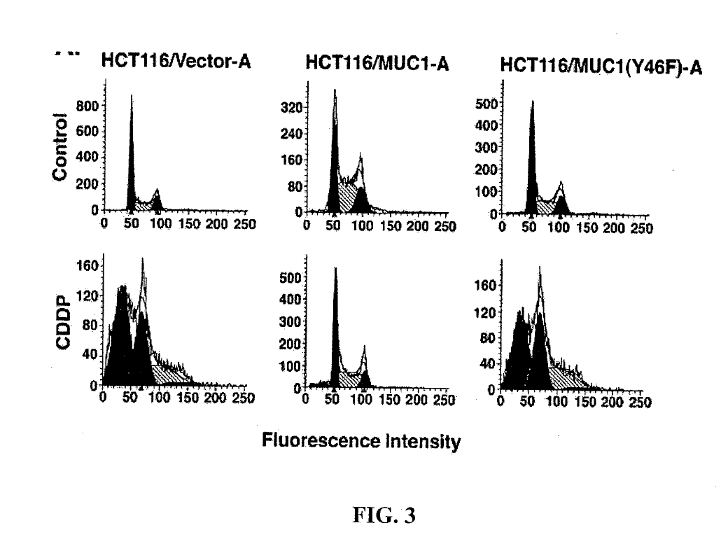

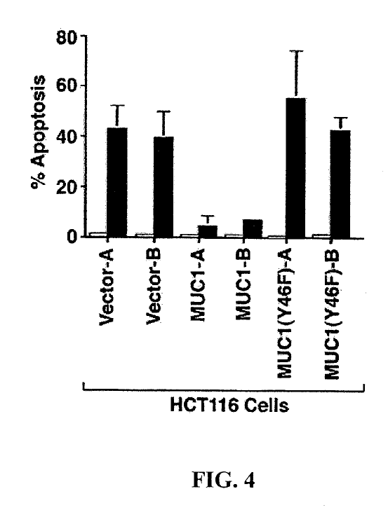

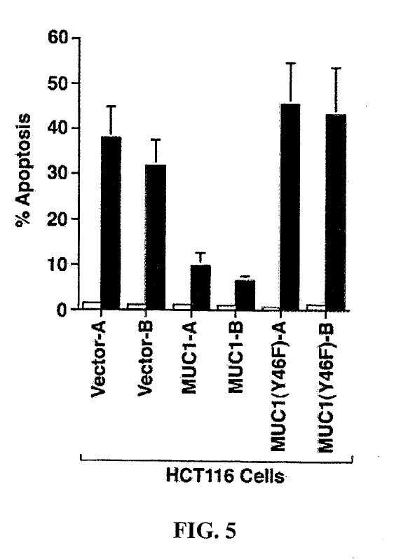

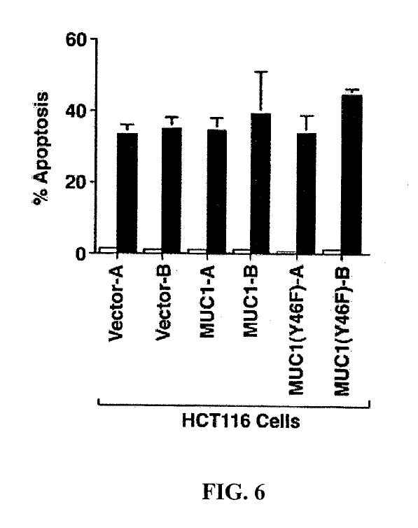

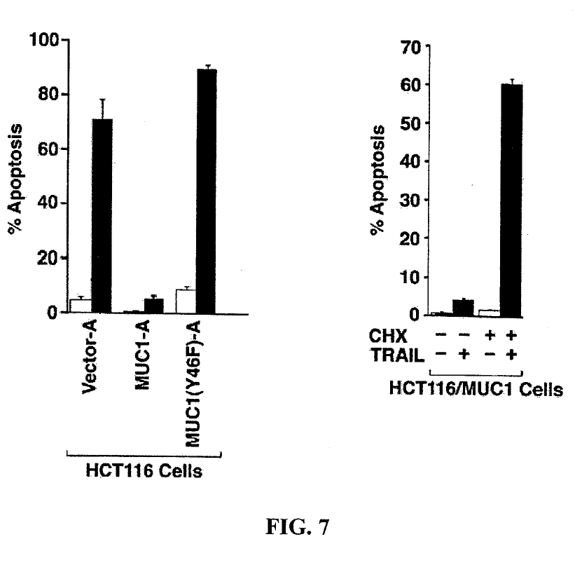

Results. To determine if MUC1 affects the induction of apoptosis by CDDP, cells were analyzed for sub-G1 DNA content. Treatment of HCT116/vector cells with CDDP for 24 h was associated with approximately 40% apoptosis (FIG. 3). Significantly, CDDP-induced apoptosis was attenuated in HCT116/MUC1, but not in HCT116/MUC1(Y46F), cells (FIG. 3). The attenuation of apoptosis by MUC1 as determined by cells with sub-G1 DNA content was confirmed when using TUNEL staining as an alternative method. In addition, similar results were obtained in multiple experiments with the separately isolated HCT116 cell clones (FIG. 4). Expression of wild-type MUC1, but not the MUC1(Y46F) mutant, also blocked apoptosis induced by the genotoxic agent, etoposide (FIG. 5). Stimulation of cell surface death receptors with TNF-α or the TNF-related apoptosis inducing factor TRAIL is associated with activation of the extrinsic apoptotic pathway. To determine if MUC1 affects death receptor-induced apoptosis, HCT116 cells were treated with TNF-α. In concert with previous work (Tsuchida et al., 1995), TNF-α alone failed to induce apoptosis of HCT116/vector cells. However, treatment with TNF-α in the presence of cycloheximide (CHX) was associated with induction of HCT116/vector cell apoptosis (FIG. 6). Similar results were obtained when HCT116/MUC1 and HCT116/MUC1(Y46F) cells were treated with TNF-α and CHX (FIG. 6), indicating that MUC1 has no effect on TNF-α+CHX-induced apoptosis. By contrast, TRAIL was effective in inducing apoptosis of HCT116/vector cells without adding CHX and, importantly, MUC1, but not MUC1(Y46F), attenuated this response (FIG. 7). Moreover, when HCT116/MUC1 cells were treated with TRAIL+CHX, MUC1 was ineffective in attenuating TRAIL-induced apoptosis (FIG. 7). Of note, CHX had no apparent effect on expression of MUC1 C-ter. These findings indicate that i) mitochondrial localization of MUC1 attenuates apoptosis induced by activation of the intrinsic pathway and ii) MUC1 attenuates TRAIL-induced apoptosis by a mechanism that may be mediated by a short-lived protein.

### REFERENCES

Adams et al., Science, 281:1322-1326, 1998.
Ashkenazi et al., J. Clin. Invest., 104:155-162, 1999.
Ashkenazi & Dixit, Science, 281, 1322-1326, 1998.
Ashkenazi & Dixit, Curr. Opin. cell Biol., 11:255-260.1999.
Bellgrau et al., Nature, 377:630-632, 1995.
Bodmer et al., J. Biol. Chem., 275, 20632-20637, 2000.
Boldin et al., Cell 85:803-815. 1996
Bunz, 1:337-341, 2001.
Burns & El Deiry, J. Biol. Chem., 276, :37879-37886, 2001.
Datta et al., Cell Growth Differ 6:363-370, 1995.
Deng et al., Genes Dev., 16:33-45, 2002.
Du et al., Cell, 102:32-42, 2000.
Emoto et al., EMBO 14:6148-6156, 1995.
French & Tschopp, J. Exp. Med., 190:891-893, 1999.
Gendler et al., J Biol Chem 263:12820-12823, 1988.
Griffith et al., Immunity, 5:7-16, 1996.
Gross et al., J. Biol. Chem., 274:1156-1163, 1999.
Herr & Debatin, Blood 98:2603-2614.
Hunt & Evans, Science, 293: 1784-1785, 2001.
Hymowitz et al., Mol. Cell., 4:563-571, 1999.
Irmler et al., Nature, 388:190-195, 1997.
Jattela et al., Oncogene, 10:2297-2305, 1995.
Kataoka et al., J. Immunol., 161:3936-3942, 1998.
Kayagaki et al., J. Exp Med., 182:1777-1783, 1995.
Kharbanda et al., Cancer Res., 56:3617-3621, 1996.
Kischkel, EMBO, 14:5579-5588, 1995.
Kluck et al., Science 275:1132-1136, 1997.
Kroemer & Reed, Nat Med 6:513-519, 2000.
Kufe, Hybridoma 3:223-232, 1984.
Kumar et al., Mol. Pharmacol., 63:276-282, 2003.
LeBlanc et al., Nat. med., 8:274-281, 2002.
Li et al., Cell 91:479-489, 1997.
Li et al., Cell, 94:491-501, 1998.
Li et al., Mol Cell Biol 18, 7216-7224, 1998a.
Li et al., J Biol Chem 276:6061-6064, 2001.
Li et al., J Biol Chem 276:35239-35242, 2001a.
Li et al., Cancer Biol Ther 2:187-193, 2003.
Li et al., Mol Cancer Res 1:765-775, 2003a.
Li et al., Oncogene 22:6107-6110, 2003b.
Ligtenberg et al., Cancer Res., 52:223-232, 1992.
Liu et al., Cell 86:147-157, 1996.
Luo et al., Cell, 94:481-490, 1998.
Mariani et al., Eur. J. Immunol., 25:2303-2307, 1995.
Marsters et al., Curr. Biol., 7:1003-1006, 1997.
Milik et al., J. Clin. Invest., 99:1082-1091, 1997.
Muzio et al., Cell 85:817-827, 1996.
Nagata, Cell., 88:355-365, 1997.
Pan et al., Science, 276:111-113, 1997.
Pan et al., Science, 277:815-818, 1997a.
Pitti et al., J. Biol. Chem., 271:12697-12690, 1996.
Poulaki et al., Drug Res. Update, 4:233-242, 2001.
Ren et al., J Biol Chem 277:17616-17622, 2002.
Sato et al., Science, 268:411-415, 1995.
Scaffidi et al., J. Biol. Chem., 274:22532-22539, 1999.
Siddiqui et al., Proc Natl Acad Sci USA 85:2320-2323, 1988.
Srinivasula et al., Mol Cell 1:949-957, 1998.
Srinivasan et al., J. Biol. Chem., 273:4523-4529, 1998a.
Stennicke et al., J Biol Chem 273:27084-27090, 1998.
Susin et al., Nature, 397:441-446, 1999.
Tsuchida et al., J. Immunol., 154:2403-2412, 1995.
Timmer et al., J. Pathol., 196:125-134, 2002.
Verhagen et al., Cell, 102:43-53, 2000.
Vermeer et al., Nature, 422:322-326, 2003.
Walczak et al., EMBO, 16:5386-5397, 1997.
Walczak et al., Nat. Med., 5:157-163, 1999.
Wang & El-Deiry, Oncogene, 24:8628-8633, 2003.
Yang et al., Science 275:1129-1132, 1997.

1. A method of enhancing death receptor-induced apoptosis in a MUC1 expressing cell comprising contacting said MUC1 expressing cell with an effective amount of a MUC1 antagonist.

2. The method of claim 1, wherein said MUC1 expressing cell is a MUC1 expressing cancer cell.

3. The method of claim 1, wherein said death receptor-induced apoptosis is Fas-induced apoptosis.

4. The method of claim 1, wherein said death receptor-induced apoptosis is a TRAIL receptor-induced apoptosis.

5. The method of claim 1, wherein said MUC1 antagonist is an antisense polynucleotide or a siRNA polynucleotide.

6. The method of claim 1, wherein said MUC1 antagonist is a MUC1 ligand trap molecule.

7. The method of claim 1, wherein said MUC1 antagonist inhibits the binding of MUC1 to a PDZ domain.

8. The method of claim 1, wherein said MUC1 expressing cell is within a patient in need of treatment comprising induction of death receptor-induced apoptosis cell death of said MUC1 expressing cell.
